(19) United States
(12) Patent Application Publication
(10) Pub. No.: US 2026/0183232 A1
(43) Pub. Date: Jul. 2, 2026

(54) Hydration Management Product

(71) Applicant: 5-HOUR INTERNATIONAL CORPORATION PRIVATE LIMITED, Singapore (SG)

(72) Inventors: William Tally, Milford, MI (US); Jack Juni, Royal Oak, MI (US); Manoj Bhargava, Farmington Hills, MI (US)

(73) Assignee: True Social IP Holdings Limited, Athlone (IE)

(21) Appl. No.: 19/130,249

(22) PCT Filed: Nov. 17, 2023
(86) PCT No.: PCT/US23/80306

§ 371 Date: May 15, 2025

Related U.S. Application Data
(60) Provisional application No. 63/426,479, filed on Nov. 18, 2022.
(60) Provisional application No. 63/428,862, filed on Nov. 30, 2022.
(60) Provisional application No. 63/441,936, filed on Jan. 30, 2023.

(51) Int. Cl. A61K 9/00; A23B 70/10; A23L 2/38; A23L 2/54; A23L 2/60; A23L 33/16; A23L 33/175; A61K 31/194; A61K 31/198; A61K 33/14

(52) U.S. Cl. CPC A61K 9/0095; A23B 70/10; A23L 2/38; A23L 2/54; A23L 2/60; A23L 33/16; A23L 33/175; A61K 31/194; A61K 31/198; A61K 33/14

(57) ABSTRACT

This disclosure relates to compositions that help achieve or maintain a desired hydration status. In general, the compositions hydrate better than the equivalent volume of water or hydrate better than a larger volume of water. The compositions include a source of calcium, magnesium and potassium but lack sodium. The compositions of the disclosure are made with extremely pure water, preferably water made using reverse osmosis components.

This application claims the benefit of U.S. provisional patent application 63/426,479 filed Nov. 18, 2022, the benefit of U.S. provisional patent application 63/428,862 filed Nov. 30, 2022 and the benefit of U.S. provisional patent application 63/441,936 filed Jan. 30, 2023.

### INTRODUCTION

The benefits of achieving and maintaining adequate hydration have been well-established, not just for active individuals but also for more sedentary individuals. Although there are many hydration compositions, most include undesirable ingredients for some people, such as high concentrations of sodium or high concentrations of sugar. It would be beneficial to have compositions that would achieve or maintain hydration status without including undesirable ingredients.

### BRIEF DESCRIPTION OF DRAWINGS

FIG. 1 shows a graph illustrating the results of a trial comparing the hydration management properties of a compositions of the disclosure to bottled water;

FIG. 2 shows a graph illustrating the results of a trial after controlling for baseline hydration status.

### SUMMARY

This disclosure relates to compositions that help achieve or maintain a desired hydration status. In general, compositions of the disclosure hydrate better than the equivalent volume of water or hydrate better than a larger volume of water. In preferred examples, the compositions include a source of calcium, a source of magnesium and a source of potassium. In preferred examples, the compositions essentially lack sodium. The disclosed compositions may also include taurine, L-serine, or D-mannose. The compositions may include glycine. The compositions may also include flavorings, artificial sweeteners, preservatives or other ingredients. In some examples, compositions of the disclosure have an acid, such as citric acid, that results in the compositions having an acidic pH.

### DETAILED DESCRIPTION

This disclosure relates to compositions that help an individual manage hydration status. For example, compositions of the disclosure may help an individual achieve a desired hydration status. In other examples, compositions of the disclosure may help an individual maintain a desired hydration status. Compositions of the disclosure may help an individual both achieve a desired hydration status and maintain a desired hydration status.

A determination of hydration status may be subjective, wherein an individual recognizes that they are adequately hydrated according to certain self-identified parameters. For example, an adequate hydration status may be the absence of certain symptoms such as thirstiness, headaches, muscle cramps, nausea, poor skin tone and appearance, or dizziness or some combination of these symptoms. In some examples, hydration status may be measured by determining the osmolarity (or the measure the related parameter osmolality) of a bodily fluid. For example, the osmolarity of saliva may be conveniently measured non-intrusively. Although, optimal hydration status may vary from individual to individual, various organizations have suggested optimal ranges for osmolarity (or osmolality) as a measure of adequate hydration status.

In some examples, the compositions are non-carbonated. In other examples, hydration compositions of the disclosure are carbonated. According to the disclosure, hydration compositions may be stored in metal, glass, or plastic containers. The hydration compositions may be stored either at room temperature or at a lower temperature.

In general, the compositions of the disclosure allow an individual to achieve or maintain a desired hydration status more readily than consuming only water. For example, individuals may consume less (e.g. consume a smaller volume) of the disclosed compositions than water to achieve or maintain the same relative hydration status. For example, the consumption of eight ounces (about 240 ml) of a disclosed compositions may achieve the same hydration effects as 16 ounces (about 480 ml) of water, or the same effects as 12 (about 360 ml) ounces of water.

Although not wishing to be bound by theory, the composition of hydration compositions of the disclosure may increase the transport of water into cells or tissues compared to the ingestion of water alone or compared to other hydration compositions, thereby improving an individual's hydration status. The properties of the compositions of the disclosure may be beneficial in achieving and maintaining hydration status in groups or populations that are at risk of a poor hydration status. For example, hydration compositions of the disclosure may alleviate poor hydration resulting from physical activity where the hydration compositions are taken before, during or after physical activity. The hydration compositions may be beneficial when the climate affects an individual's hydration status.

Ingestion of the compositions of the disclosure may increase the uptake or bioavailability of substances taken concurrently with the hydration composition. For example, the uptake of medicines into tissues or cells may be facilitated by ingestion of a composition according to the disclosure.

An effective, single serving size of the disclosed compositions may vary with individual requirements. In particularly preferred examples, an effective single serving of a compositions may be from about six ounces (about 180 ml) to about 24 ounces (about 720 ml) of liquid having the disclosed ingredients, or from about six ounces to 18 (540 ml) ounces, or from about six ounces to about 16 (about 480 ml) ounces, or from about six ounces to about 12 (about 360 ml) ounces of liquid. In preferred examples, compositions according to the disclosure may have a serving size of about 8 ounces of liquid, or a serving size of about 12 ounces or a serving size of about 16 ounces.

In general, compositions of the disclosure include selected electrolytes. For example, compositions of the disclosure have at least two of potassium, calcium or magnesium. In preferred examples, compositions include potassium, calcium and magnesium. In particularly preferred examples, compositions of the disclosure lack sodium. That is, sodium is removed to near undetectable levels during purification of the water that is used to manufacture the compositions and a source of sodium is not added to the final composition in any form.

Sources of electrolytes may be selected on the basis of solubility in water or relative bioavailability or a combination of both solubility in water and relative bioavailability. Sources of electrolytes and other ingredients may be also selected to permit use of the compositions on an empty stomach without causing irritation to a consumer. Potassium chloride is a preferred source of potassium. Calcium citrate is a preferred source of calcium. According to the disclosure, magnesium glycinate is a preferred source of magnesium.

In preferred examples, calcium is present at a concentration of from about 0.10 mg/ml to about 1.00 mg/ml, or from about: 0.20 mg/ml to about 0.70 mg/ml, or from about 0.30 mg/ml to about 0.60 mg/ml.

In preferred examples, magnesium is present at a concentration of from about 0.10 mg/ml to about 1.00 mg/ml, or from about 0.20 mg/ml to about 0.70 mg/ml, or from about 0.30 mg/ml to about 0.60 mg/ml.

In preferred examples, potassium is present at a concentration of from about 0.25 mg/ml to about 3.00 mg/ml or from about 0.40 mg/ml to about 1.40 mg/ml, or from about 0.70 mg/ml to about 1.3 mg/ml.

In preferred examples, compositions of the disclosure include a source of chloride. Potassium chloride is a particularly preferred form of chloride. Chloride may be present from about 0.3 mg/ml to about 3.0 mg/ml, or from about 0.7 mg/ml to about 1.5 mg/ml, or from about 0.8 mg/ml to about 1.2 mg/ml.

According to the disclosure, the selected electrolytes may provide one or more benefits to a consumer. For example, according to the disclosure, calcium positively affects bone health. Magnesium also help positively affects bone health. In other examples, magnesium may have positive effects on the mood of a user, such as reducing anxiety. Magnesium may also positively affect blood sugar control. Magnesium may also act as an anti-inflammatory or may reduce leg cramps. According to the disclosure, magnesium may help provide one or more of the disclosed benefits to a user. Potassium may lower blood pressure, improve muscle function, improve nerve function, reduce muscle cramps, or lower blood pressure, or potassium may provide some combination of these benefits. According to the disclosure, chloride regulates electrolyte balance in cells. Chloride may also positively affect digestion.

In preferred examples, taurine is included in the disclosed compositions. In preferred examples, taurine is included at a concentration of from about 1.0 mg/ml to 3.0 mg/ml. In particularly preferred examples, taurine is present at a concentration of about 1.5 mg/ml to 2.5 mg/ml. Taurine is believed to influence electrolyte balance in cells. Taurine may also improve nocturnal leg cramps, or may delay muscle fatigue, or may have anti-inflammatory effects, may positively affect blood sugar levels, or may have positive effects on mood. In particularly preferred examples, taurine may provide a combination of the disclosed benefits.

In preferred examples, L-serine is included in the disclosed compositions. In preferred examples, L-serine is present at a concentration of from about 1.0 mg/ml to 3.0 mg/ml. In particularly preferred examples, L-serine is present at a concentration of about 1.5 mg/ml to 2.5 mg/ml. L-serine influences electrolyte balance in cells. L-serine may also positively affect mood, sleep, memory or digestion or some combination of these benefits.

In preferred examples, the disclosed compositions Include glycine. In preferred examples, glycine is present at a concentration of from about 0.5 mg/ml to about 5.0 mg/ml. In particularly preferred examples, glycine is present at a concentration of about 3 mg/ml to 5.0 mg/ml. In particularly preferred examples, glycine is from magnesium glycinate. Glycine may positively affect mood, sleep quality, may lower blood pressure, may improve a user's response to insulin, or may decrease oxidative stress or may provide some combination of these benefits.

In some examples, compositions according to the disclosure include D-mannose. D-mannose may positively affect urinary tract health, such as prevent urinary tract infections. D-mannose may be present at a concentration of from about 0.10 mg/ml to about 1.0 mg/ml.

In preferred examples, compositions of the disclosure use extremely pure water to form the compositions. In preferred examples, water used for the compositions has a conductivity of less about 10 μS/cm (microsiemens per centimeter), or less than about 7 μS/cm, or less than about 4 μS/cm, or has a conductivity of about 1 μS/cm, when the conductivity is measured before the addition of composition components. In preferred examples, the conductivity of the water used for use in the compositions has a conductivity of 1 μS/cm to 10 μS/cm, or from about 1 μS/cm to about 4 μS/cm.

In preferred examples, water for forming or making up the volume the compositions is purified by using reverse osmosis procedures. In preferred examples, water may flow through multiple reverse osmosis components placed in series to achieve the required purity. For example, water may be flowed through two or more water treatment systems, each having reverse osmosis components, where the reverse osmosis systems are placed in series. That is, permeate produced by a first reverse osmosis water treatment is flowed to a second water treatment system for further treatment, producing a second permeate. The purity of the second permeate is generally adequate for use for compositions of the disclosure. In some examples, a second permeate produced from a second water treatment system is flowed to a third water treatment system. In other examples, the reverse osmosis components may be placed in series within the same self-contained system. Although not wishing to be bound by theory, the use of highly purified water may reduce or prevent adverse effects or reactions due to the presence of undesirable components in the water used during manufacturing. In other examples, water for compositions may be purified by other water purification methods or a combination of reverse osmosis and other methods, including the use of de-ionizing resins, to achieve the required purity.

According to the disclosure, compositions of the disclosure may include an artificial sweetener. Sucralose is a preferred artificial sweetener. In other examples, sucralose may not be added. In preferred examples, sucralose is present from 0.05 mg/ml to about 0.3 mg/ml. Compositions of the disclosure may include natural or artificial flavoring ingredients, such as fruit flavorings. For example, compositions of the disclosure may include natural lemon flavoring.

According to the disclosure, compositions may include at least one preservative. In preferred examples, potassium sorbate is used as a preservative. In some examples, potassium sorbate is present at a concentration of from about 0.1 mg/ml to about 0.5 mg/ml.

According to the disclosure, the pH of compositions may be basic. In some examples, the pH of a compositions may be from about 7.0 to about 9.0. In particularly preferred examples, the pH of the compositions is from about 8.0 to about 9.0. In preferred examples, the pH of the compositions is from about 8.8 to about 9.2.

In other examples, compositions may be acidic. In some examples, compositions with an acidic pH have a reduced risk of microbial contamination. For example, acidic compositions may not require refrigeration. In preferred examples, the pH may be from about from 4.0 to about 5.0. In particularly preferred examples, the pH is from about 4.0 to about 4.8. In particularly preferred examples, the pH is from about 4.0 to about 4.4. In preferred examples, the pH is below about 4.6. In some examples, compositions of the disclosure may include phosphoric acid. In particularly preferred examples, the pH is adjusted to a desired acidic pH using organic or naturally occurring acids or a combination of organic or naturally occurring acids. In particularly preferred examples, the added acid is citric acid. In other examples, the added malic acid or a combination malic acid and citric acid. In some examples an organic acid may be present from about 2.5 mg/ml to 6.0 mg/ml.

The osmolality of the compositions of the disclosure vary with the ingredients added but are generally in the range of 120 to 220 mOsmol/L.

In preferred examples, compositions of the disclosure provide benefits relating to the physiological status of an individual, such as benefits related to the health of an individual. In some examples, physiological or health benefits may derive from improved hydration management when using compositions according to the disclosure. For example, the compositions may provide benefits related to control of blood pressure, control of blood sugar, or control of weight loss. In preferred examples, the benefits may accrue to individuals having normal physiological parameters relating to control of blood pressure, control of blood sugar, or control of weight loss. In other examples, benefits may accrue to individuals having one or more physiological parameters that are considered abnormal relating to control of blood pressure, control of blood sugar, or control of weight loss. In preferred examples, benefits of the disclosed compositions may accrue to older individuals. In some examples, compositions of the disclosure may promote or maintain skin health or skin appearance.

Results of trials with the disclosed compositions show that the disclosed compositions achieve and maintain hydration status better than water. For example, in one example, individuals were given either an eight-ounce serving of a composition according to the disclosure or were given eight ounces of water. The individuals were monitored for hydration status by measuring weight loss or by measuring the osmolarity of saliva at various timepoints after taking the samples. Individuals given the compositions showed better hydration status, as determined by the values of the measured parameters.

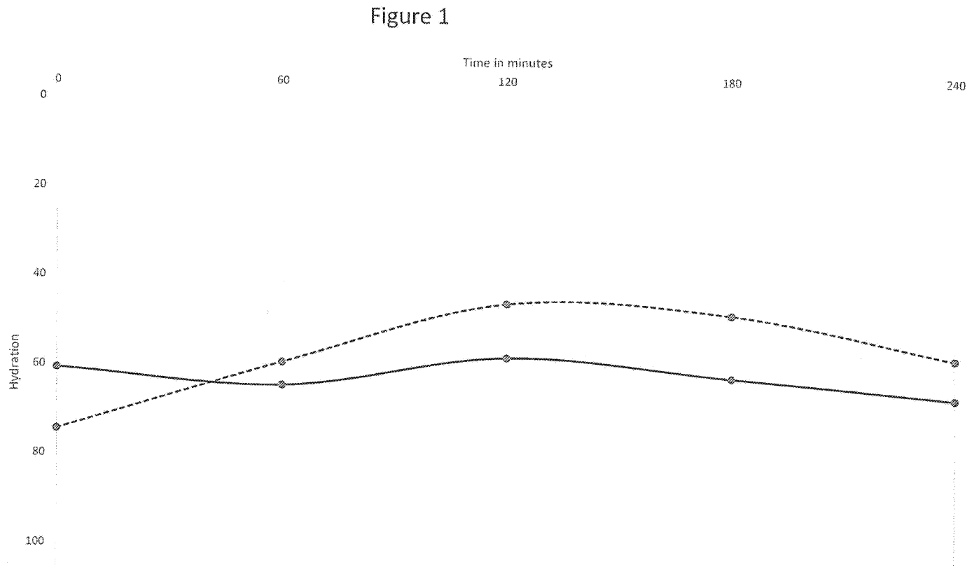

FIG. 1 shows the results of trial for testing the hydration properties of a compositions according to the disclosure. In this example of a crossover study, ten individuals were first given either 16 ounces of a bottled water composition or 12 ounces of a composition according to the disclosure. At selected timepoints after consumption, hydration status was determined by measuring the osmolarity (mOsm) of saliva using an MX3 hydration testing system. After a washout period, the ten individuals were given the other test sample, and the same testing procedure repeated.

FIG. 1 shows the plots of average hydration status (mOsm; vertical axis) at selected timepoints (minutes; horizontal axis) for either the 12 ounces compositions of the disclosure (dashed plot) or 16 ounces of bottled water (solid plot) over the course of the trial. The 12 ounces compositions performed significantly better than 16 ounces of water in this trial with respect to hydration status as measured by the osmolarity of saliva. In this Figure, improved hydration status is indicated by a plot trending upward.

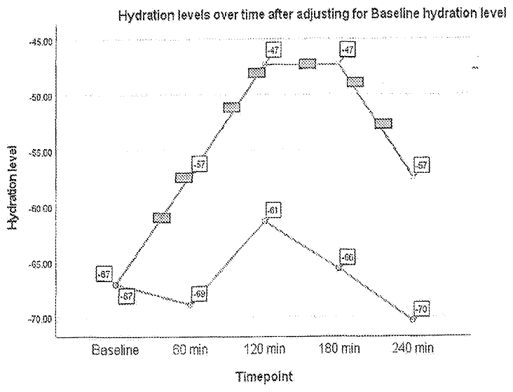

FIG. 2 shows a plot of the data after an analysis of covariance and controlling for baseline hydration levels. There was a significant interaction between drink type and timepoint indicating the 12 ounces composition of the disclosure (line with filled rectangles) according to the disclosure performed significantly better than 16 ounces bottled water (no rectangles) over time (p<0.01). A comparison of drink groups up to 120 minutes reveals between group differences with 12 ounces of the hydration composition significantly outperforming 16 ounces of water at both the 60 and 120 minute timepoints (p=0.040 and p=0.048 respectively).

Tables 1-5 disclose non-limiting examples of compositions according to the disclosure.

TABLE 1

| 12 ounces | mg per |
|---|---|
| serving | serving |
| Calcium | 120-200 |
| Magnesium | 120-200 |
| Chloride | 300-400 |
| Potassium | 300-400 |
| Taurine | 600-800 |
| L-Serine: | 600-800 |
| Glycine | 1000-1400 |
| D-Mannose | 120-200 |
| Potassium Sorbate | 72 |
| Sucralose | 0 |
| citric acid | 1500 |

TABLE 2

| 12 ounces | mg per |
|---|---|
| serving | serving |
| Calcium | 120-200 |
| Magnesium | 120-200 |
| Chloride | 300-400 |
| Potassium | 300-400 |
| Taurine | 600-800 |
| L-Serine | 600-800 |
| Glycine (from | 600-800 |
| Magnesium Glycinate) | |
| D- Mannose | 120-200 |
| Potassium Sorbate | 72 |
| sucralose | 30 |
| citric acid | 1500 |

TABLE 3

| 12 ounces | mg per |
|---|---|
| serving | serving |
| Calcium | 120-200 |
| Magnesium | 120-200 |
| Chloride | 300-400 |
| Potassium | 300-400 |
| Taurine | 600-800 |
| L-Serine | 600-800 |
| Glycine (from | 600-800 |
| Magnesium Glycinate) | |
| Potassium Sorbate | 72 |
| Sucralose | 0 |
| citric acid | 1500 |

TABLE 4

| Test 12 ounces | mg per |
|---|---|
| serving | serving |
| Calcium (As Calcium Citrate) | 120-200 |
| Magnesium (as Magnesium glycinate) | 120-200 |
| Chloride (from Potassium Chloride) | 300-400 |
| Potassium (As Potassium Chloride) | 300-400 |
| Taurine | 600-800 |
| L-Serine | 600-800 |
| Glycine (from Magnesium Glycinate) | 600-800 |
| Potassium Sorbate | 72 |
| sucralose | 30 |
| citric acid | 1500 |

TABLE 5

| | Amount |
|---|---|
| | in 8 ounces |
| Ingredient | (mg) |
| Magnesium | 100 |
| Calcium | 100 |
| Potassium | 290 |
| Taurine | 500 |
| L-Serine | 500 |
| D-mannose | 100 |

We claim:

1. A composition comprising
potassium, said potassium at a concentration of 0.10 mg/ml to about 1.00 mg/ml;
calcium said calcium at a concentration of 0.10 mg/ml to about 1.00 mg/ml
magnesium said magnesium at a concentration of about 0.25 mg/ml to about 3.00 mg/ml;
chloride;
L-serine;
taurine;
glycine;
citrate; and
said composition having a liquid volume of from about 8 ounces to 16 ounces.

2. The composition of claim 1, wherein a source of said potassium is potassium chloride.

3. The composition of claim 1, wherein a source of said calcium is calcium citrate.

4. The composition of claim 1, wherein a source of said magnesium is magnesium glycinate.

5. The composition of claim 1, wherein a pH of said composition is from about 7.0 to about 9.0.

6. The composition of claim 1, wherein a source of said citrate is both calcium citrate and citric acid.

7. The composition of claim 1, wherein a source of said citrate is both calcium citrate malate and citric acid.

8. The composition of claim 7, wherein a pH of said composition is from about 3.0 to about 6.0

9. The composition of claim 1, further comprising an artificial sweetener.

10. The composition of claim 1, further comprising a preservative.

11. The composition of claim 10, wherein said preservative is potassium sorbate.

12. The composition of claim 1 wherein said composition uses water produced by at least two reverse osmosis water treatment systems, wherein said at least two reverse osmosis water purification systems are placed in series.

13. The composition of claim 12, wherein said water has a conductivity of from about 1 μs/cm to about 10 μs/cm.

14. The composition of claim 13, wherein said water has a conductivity from about 1 μs/cm to about 4 μs/cm.

15. The composition of claim 1, wherein said taurine is present at from 1.0 mg/ml to about 3.0 mg/ml.

16. The composition of claim 1, wherein said L-serine is present rom about 1.0 mg/ml to about 3.0 mg/ml.

17. The composition of claim 1, further comprising citric acid at a concentration of 2.5 mg/ml to 6.0 mg/ml.

18. The composition of claim 17, wherein a pH of said composition is from about 4.0 to about 4.6.

19. The composition of claim 1, wherein said chloride is present from about 0.3 mg/ml to about 3.0 mg/ml.

20. The composition of claim 1 wherein said composition is carbonated.
